(19) United States
(12) Patent Application Publication
(10) Pub. No.: US 2015/0307412 A1
(43) Pub. Date: Oct. 29, 2015

(54) METHODS AND APPARATUSES FOR PRODUCING XYLENE FROM PROPYLBENZENE

(71) Applicant: UOP LLC, Des Plaines, IL (US)

(72) Inventors: Patrick Whitchurch, Sleepy Hollow, IL (US); Stanley Joseph Frey, Palatine, IL (US)

(73) Assignee: UOP LLC, Des Plaines, IL (US)

(21) Appl. No.: 14/260,185

(22) Filed: Apr. 23, 2014

(51) Int. Cl. C07C 1/22; C07C 7/00; C07C 7/13; C07C 6/06; C07C 5/27

(52) U.S. Cl. CPC C07C 1/22; C07C 6/06; C07C 5/2732; C07C 7/13; C07C 7/00

(57) ABSTRACT

Methods and apparatuses are provided for producing xylene. A method includes combining a propylbenzene containing feed with a xylene raffinate stream, where the xylene raffinate stream is provided from a xylene recovery unit. The xylene raffinate stream and the propylbenzene containing feed are isomerized in an isomerization unit to produce an xylene isomerization effluent stream, where the xylene isomerization effluent stream includes aromatic compounds having 8, 9, or 10 carbons atoms. The aromatic compounds having 8 carbon atoms are separated from the aromatic compounds having 9 or 10 carbons, and the aromatic compounds having 8 carbons are fed to the xylene recovery unit. The aromatic compounds having 9 or 10 carbons are transalkylated with toluene to produce xylene.

### TECHNICAL FIELD

The present disclosure generally relates to systems and methods for producing xylene from propylbenzene, and more particularly relates to systems and methods for recovering a desired isomer of xylene from propylbenzene.

### BACKGROUND

Xylene isomers are important intermediates in chemical syntheses, and specific xylene isomers are desired for different processes. Paraxylene is a feedstock for terephthalic acid production, and terephthalic acid is used in the manufacture of synthetic fibers and resins. Metaxylene is used in the manufacture of certain plasticizers, azo dyes, and wood preservatives. Orthoxylene is a feedstock for phthalic anhydride production, and phthalic anhydride is used in the manufacture of certain plasticizers, dyes, and pharmaceutical products. Desired xylene isomers are typically extracted from petroleum feedstocks, but the increasing demand for specific xylene isomers creates supply pressures on the petroleum feedstocks.

An aromatics complex may include a transalkylation process unit. The transalkylation process unit can increase the yield of xylenes in the aromatics complex because it converts toluene and aromatics with 9 carbon atoms into 8 carbon xylene compounds. However, alkyl groups with 2 or more carbon atoms, such as ethyl, propyl and butyl groups, tend to be severed from benzene compounds during transalkylation such that benzene and naphthenes or paraffins are produced. Benzene, naphthenes, and paraffins have a lower value than xylenes.

Propylbenzene and other higher alkyl benzene compounds, such as ethylbenzene and butylbenzene, are available from certain sources. For example, reformate from naphtha feedstocks may include high concentrations of higher alkyl benzene compounds, including propylbenzene. Higher alkyl benzene compounds include alkyl groups with 2 or more carbons attached to a benzene ring, such as ethylbenzene, propylbenzene, or butylbenzene. Propylbenzene and other higher alkyl benzene compounds are also available in depolymerized lignin. Lignin is a polymer produced in large quantities by woody plants. Lignin is a solid, but it can be solubilized and depolymerized to produce liquid products. There are typically several different compounds in liquid products produced from lignin, including high concentrations of higher alkyl benzene compounds, but depolymerized lignin includes relatively small quantities of benzene with attached methyl groups. As such, transalkylation of the higher alkyl benzene compounds from depolymerized lignin tends to produce benzene, naphthenes, and paraffins with relatively low production of xylene compounds.

Accordingly, it is desirable to develop apparatuses and methods for converting higher alkyl benzenes into xylenes. In addition, it is desirable to develop apparatuses and methods for producing xylenes from reproducible natural feedstocks, such as woody plants. Furthermore, other desirable features and characteristics of the present embodiment will become apparent from the subsequent detailed description and the appended claims, taken in conjunction with the accompanying drawings and this background.

### BRIEF SUMMARY

Methods and apparatuses for producing xylene are provided herein. In an embodiment, a method is provided for producing xylene that includes combining a propylbenzene containing feed with a xylene raffinate stream, where the xylene raffinate stream is provided from a xylene recovery unit. The xylene raffinate stream and the propylbenzene containing feed are isomerized in an isomerization unit to produce an xylene isomerization effluent stream, where the xylene isomerization effluent stream includes aromatic compounds having 8, 9, or 10 carbons atoms. The aromatic compounds having 8 carbon atoms are separated from the aromatic compounds having 9 or 10 carbons, and the aromatic compounds having 8 carbons are fed to the xylene recovery unit. The aromatic compounds having 9 or 10 carbons are transalkylated with toluene to produce xylene.

In an alternate embodiment, a method of producing xylene includes isolating a desired xylene isomer from a first xylene column stream in a xylene recovery unit, and producing a xylene raffinate stream. The xylene raffinate stream and a propylbenzene containing feed are contacted with an isomerization catalyst at isomerization conditions to produce a xylene isomerization effluent stream including aromatic compounds having 8, 9, or 10 carbons atom. The aromatic compounds having 8 carbon atoms are separated from the aromatic compounds having 9 or 10 carbon atoms, and the aromatic compounds having 9 or 10 carbon atoms are contacted with a transalkylation catalyst at transalkylation conditions in the presence of toluene to produce xylene.

In another embodiment, an apparatus is provided for producing xylene. The apparatus includes a xylene recovery unit configured to isolate a desired xylene isomer from a first xylene column stream. An isomerization unit is coupled to the xylene recovery unit, and a propylbenzene containing feed is also coupled to the isomerization unit. A transalkylation unit is configured to transalkylate aromatic compounds having 9 or more carbon atoms with toluene to produce xylene. A xylene column is coupled to the xylene recovery unit, where the xylene column is configured to transfer the first xylene column stream to the xylene recovery unit and to transfer a second xylene column stream to the transalkylation unit. A fractionation zone is coupled to the transalkylation unit and to the xylene column, where the fractionation zone is configured to transfer aromatic compounds having 8 or more carbons to the xylene column.

### BRIEF DESCRIPTION OF THE DRAWINGS

The present embodiment will hereinafter be described in conjunction with the following drawing figures, wherein like numerals denote like elements, and wherein:

FIG. 1 is a schematic diagram of an exemplary embodiment of a method and apparatus for depolymerizing lignin;

FIG. 2 is an illustration of an exemplary embodiment of a lignin polymer and propylbenzene; and

FIG. 3 is a schematic diagram of an exemplary embodiment of a method and apparatus for producing xylene from propylbenzene.

### DETAILED DESCRIPTION

The following detailed description is merely exemplary in nature and is not intended to limit the application and uses of the embodiments described. Furthermore, there is no intention to be bound by any theory presented in the preceding background or the following detailed description.

The various embodiments described herein relate to methods and apparatuses for producing a desired xylene isomer from a propylbenzene containing feed, where the propylbenzene containing feed includes propylbenzene, an aromatic compounds with 9 carbons, and where about 50 mole percent or more of the aromatic compounds with 9 carbon atoms may be propylbenzene. The propylbenzene containing feed may come from various sources, such as depolymerized lignin, reformate from naphtha feedstocks, or other sources. An aromatics complex includes a xylene recovery unit configured to separate a desired xylene isomer from a first xylene column stream, and the xylene recovery unit is also configured to produce a xylene raffinate stream that includes the remaining xylene isomers. The xylene raffinate stream is fed to an isomerization unit, and the propylbenzene containing feed is also fed to the isomerization unit. The isomerization unit includes an isomerization catalyst that produces methylated aromatic compounds from higher alkyl compounds, such as from ethyl benzene, propyl benzene, and butyl benzene. The isomerization unit produces a xylene isomerization effluent stream that includes mixed xylene isomers and methylated aromatic compounds with 9 or more carbon atoms, as well as various other hydrocarbons. The xylene isomerization effluent stream is fractionated to recover a first xylene column stream that is re-introduced into the xylene recovery unit, and an A9-10 stream that includes the aromatic compounds having 9 or 10 carbon atoms. Many of the aromatic compounds having 8 or more carbon atoms are methylated in the isomerization unit, so many of the aromatic compounds having 9 or 10 carbon atoms are methylated. The A9-10 stream is transferred to a transalkylation unit with a toluene stream to produce additional xylene.

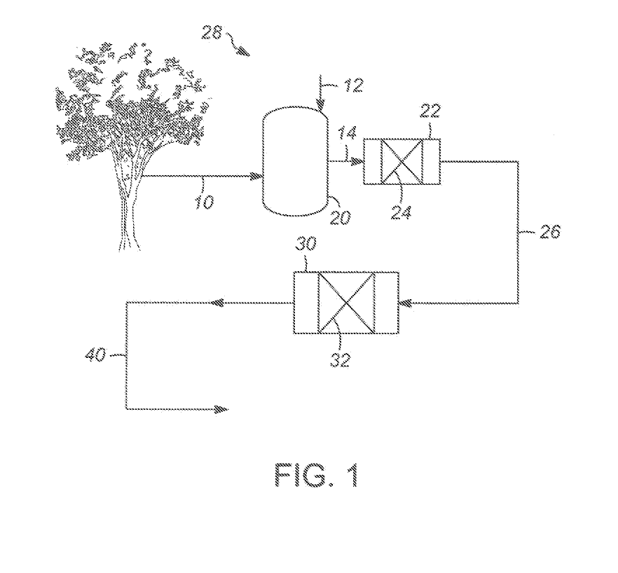

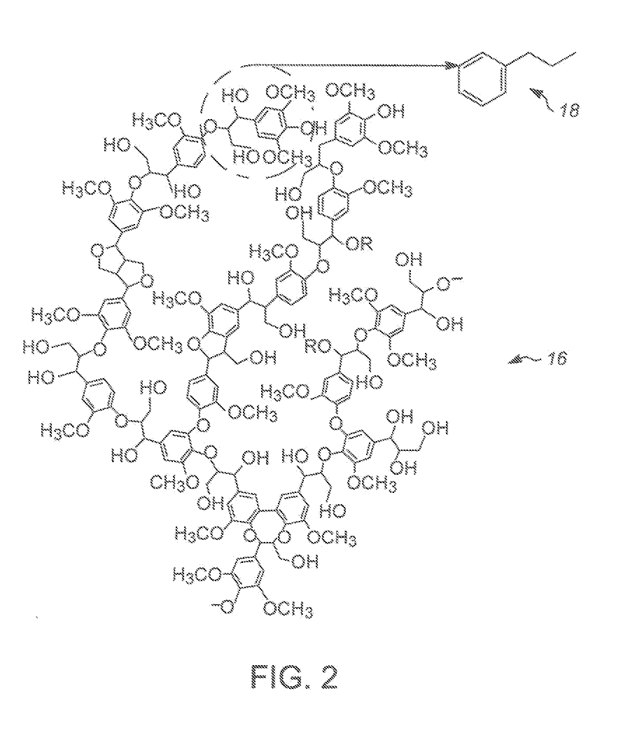

An exemplary method and apparatus will now be described with reference to FIG. 1. In this embodiment, the propylbenzene containing feed 40 is produced by depolymerizing a lignin supply 10. The lignin supply 10 is combined with a lignin solvent 12 to create a lignin slurry 14. Lignin for the lignin supply 10 is most commonly available from wood, which is a naturally occurring and renewable resource, but it is also present in the secondary cell walls of many plants and some algae, such as straw, corn stover, and bagasse. Lignin may be obtained from paper mills, saw mills, farm harvests, or a wide variety of other sources. Referring momentarily to FIG. 2, with continuing reference to FIG. 1 and without intending to be limited, an example of the polymeric form of lignin 16 is illustrated. FIG. 2 further illustrates how removal of oxygen atoms from lignin 16 during depolymerization can produce propyl benzene 18. In general, lignin is a polymer including para-hydroxyphenyl units, syringyl units, and guaiacyl units.

The lignin solvent 12 may be water in a liquid form, and the lignin solvent 12 may also include a hydrogen donor such as acids, glycols, other alcohols, or other hydrogen donors, including but not limited to ethylene glycol, propylene glycol, other glycols, sulfuric acid, hydrochloric acid, nitric acid, acetic acid, citric acid, and formic acid. In an exemplary embodiment, the lignin solvent 12 includes about 50 mass percent water or more. Many other materials can be used as the lignin solvent 12 in other embodiments, such as aromatic hydrocarbons, including but not limited to benzene, toluene, xylenes and/or fused ring aromatic compounds such as tetralin and its alkylated derivatives. Additional compounds may also be optionally added to the lignin slurry 14, such as surfactants or emulsifying agents. In some embodiments, the lignin 16 is dissolved in the lignin solvent 12 to produce a solution, but in many embodiments some of the lignin 16 is not completely dissolved to produce the lignin slurry 14.

In some embodiments, the lignin supply 10 is pretreated (not illustrated), such as with steam explosion, hammer milling, grinding, chipping or other size reduction, soaking in the lignin solvent 12 or in other solvents, saturation with steam, pressurizing at pressures from about 1 atmosphere to about 150 atmospheres, enzymatic hydrolysis, alkaline wet oxidation, other methods, or combinations thereof. Preliminary soaking may be at elevated temperatures in some embodiments, such as from about 120 degrees centigrade (° C.) to about 250° C., and may last from about 1 minute to about 24 hours or longer. The lignin 16 is often present with other compounds in plants, such as cellulose and hemicellulose, and the lignin 16 may or may not be separated from other components in the pretreatment step or steps.

The lignin slurry 14 may be formed in a lignin slurry vessel 20. In some embodiments, the particle size in the lignin slurry vessel 20 may be reduced to about 200 microns or less, such as with steam explosion or with high shear forces. The particle size may be reduced during the pretreatment step, if conducted, or in the lignin slurry vessel 20. In other embodiments, larger particle sizes are allowed. The particle size of the lignin slurry 14 may be reduced before or after adding the lignin solvent 12 to the lignin supply 10 in the lignin slurry vessel 20, and a partial vacuum may be applied to the lignin supply 10 to aid in dispersing solids in the lignin solvent 12 for particle size reduction.

The lignin slurry 14 is transferred to a lignin reactor 22 containing a lignin catalyst 24, where the lignin slurry 14 is depolymerized to form a lignin aromatic stream 26. The lignin slurry vessel 20 and the lignin reactor 22 form a lignin depolymerization unit 28, but the lignin depolymerization unit 28 may have other vessels in alternate embodiments. Hydrogen is added to the lignin slurry 14 in the presence of the lignin catalyst 24, and the hydrogen reacts with oxygen in the polymeric lignin 16 to break the carbon/oxygen bonds and thereby depolymerize the lignin 16. The hydrogen may be introduced to the lignin reactor 22 by a variety of methods or techniques, including hydrogen donor compounds in the lignin slurry 14 or as gaseous hydrogen. The lignin reactor 22 may be more than one reactor in some embodiments, and there may be more than one type of lignin catalyst 24 used in the same or different reactors. Different lignin reactors 22 may be operated in series or in parallel, and there may be different temperatures, pressures, and residence times in different reactors as well.

The lignin slurry 14 is contacted with hydrogen in the presence of the lignin catalyst 24 under conditions which promote the depolymerization of lignin to aromatic oxygenate substituents (e.g., phenol, and its alkoxylated derivatives) and also the deoxygenation (or hydrodeoxygenation) of these substituents or intermediates to aromatic hydrocarbons. The lignin catalyst 24 may be present in the form of solid particles with a catalytically active metal disposed on a support, or in the form of a compound containing the catalytically active metal. The lignin catalyst 24 may include at least one IUPAC Group 8, 9, or 10 metal for a hydrogenation function, such as iron, cobalt, nickel, or combinations thereof, and/or one or more IUPAC Group 6 metals, such as molybdenum and tungsten. Noble metals, such as ruthenium, palladium, and platinum, may also be used for a hydrogenation function. A representative lignin catalyst 24 includes a metal selected from ruthenium, rhodium, platinum, palladium, iron, cobalt, nickel, molybdenum, tungsten, and mixtures thereof, and may include a support in some embodiments. The lignin catalyst 24 may also include an acid function, and the acid function may be imparted by the support or otherwise incorporated into the lignin catalyst 24. Representative acidic support materials that can serve as the acid function include clays (e g , minugel®, kaolin, kaolinites, halloysite, etc.), zeolites, non-zeolitic molecular sieves, mixed metal oxides, sulfated zirconia, and other materials that contain acid sites and that can be used in varying amounts to regulate the overall acidity of the catalyst. In alternate embodiments, the lignin catalyst 24 may be an acid, sodium hydroxide or other bases, or combinations of catalytically active metals with an acid or base. The lignin catalyst 24 may be in a fixed bed or moving bed, and homogeneous systems operating with catalysts that are soluble in the reactants and products may also be used.

Catalytic depolymerization/deoxygenation conditions will vary depending on the quality of the lignin conversion effluent desired, with higher severity operations resulting in greater conversion of organic oxygenate intermediates and other oxygenated species (e.g., carboxylic acids) by hydrogenation. Typical lignin depolymerization reaction conditions include a temperature of from about 40° C. to about 700° C., or from about 200° C. to about 600° C., or from about 190° C. to about 370° C. in various embodiments. If hydrogen gas is used to supply the hydrogen, the hydrogen partial pressure may be from about 6 atmospheres to about 300 atmospheres, or from about 80 atmospheres to about 250 atmospheres, or from about 50 atmospheres 150 atmospheres in various embodiments. If a hydrogen donor is used to supply the hydrogen, the hydrogen donor may be introduced into the lignin reactor 22 in the lignin slurry 14, or the hydrogen donor may be introduced separately, or the hydrogen donor may be introduced with the lignin slurry 14 and also added separately. In some embodiments, the reaction temperature is higher than the boiling point of the lignin solvent 12 at atmospheric pressure, but the reaction temperature is less than the critical temperature of the lignin slurry 14. The pressure in the reactor may be increased until the reaction proceeds without the formation of char, or carbonaceous residue, in the lignin reactor 22.

In addition to pressure and temperature, the residence time of the lignin slurry 14 and hydrogen in the lignin reactor 22 can also be adjusted to increase or decrease the reaction severity and consequently the quality of the resulting lignin aromatic stream 26. With all other variables unchanged, lower residence times are associated with lower reaction severity. For example, A weight hourly space velocity (WHSV, expressed in units of hr⁻¹) from about 0.1 hr⁻¹ to about 20 hr⁻¹ may be used, but in alternate embodiments a WHSV of from about 0.5 hr⁻¹ to about 10 hr⁻¹ may be used. The quantity of hydrogen used may be based on the stoichiometric amount needed to completely convert the oxygen present in the lignin 16 to water (H₂O). In representative embodiments, the reaction may be carried out in the presence of hydrogen in an amount ranging from about 90% to about 600% of the stoichiometric amount.

The polymeric form of lignin 16 does not have a specific form, but does include para-hydroxyphenyl units, syringyl units, and guaiacyl units, as mentioned above. As such, the depolymerized and deoxygenated compounds in the lignin aromatic stream 26 may include a relatively high concentration of N-propyl benzene compounds, and also tends to include other higher alkyl benzene compounds such as ethyl benzene and butyl benzene, as mentioned above. Propyl benzene can be referred to as an A9 compound, because it is an aromatic compound with 9 carbon atoms. In this description, “A9 ” represents aromatic compounds with 9 carbon atoms, where the letter “A” represents “aromatic” and the following number represents the number of carbon atoms in the compound. In a similar manner, A10 refers to aromatic compounds with 10 carbon atoms; “A7-” refers to aromatic compounds with 7 or fewer carbon compounds, and so on. In an exemplary embodiment, the lignin aromatic stream 26 includes several different compounds, including A8, A9, and A10 compounds, where about 50 mole percent or more of the A8 compounds are ethyl benzene, about 50 mole percent or more of the A9 compounds are n-propyl benzene, and/or about 50 mole percent or more of the A10 compounds are N-butyl benzene.

The lignin aromatic stream 26 may optionally be fractionated in a lignin fractionator (not illustrated) to produce a fractionated lignin aromatic stream that primarily includes A9 compounds, or A9-10 compounds, or A9-11 compounds, or other selected compounds in various embodiments. The fractionated lignin aromatic stream may be further processed as described below for the lignin aromatic stream 26.

The lignin aromatics stream 26 may be treated in a hydrotreater 30, and remaining oxygenates that may be present in the lignin aromatics stream 26 are removed to produce the propylbenzene containing feed 40. As discussed above, the depolymerization of lignin 16 can proceed to varying degrees of completeness, so some oxygenates may remain on the compounds in the lignin aromatic stream 26 in some embodiments. The oxygenates are not desired in downstream processes, so they are removed with the hydrotreater 30, where the propylbenzene containing feed 40 may include oxygen at a concentration of about 10 parts per million or less by weight, where the oxygen is incorporated in hydrocarbons. Hydrotreating is a well-known process, and involves contacting the lignin aromatics stream 26 and hydrogen gas with a hydrotreating catalyst 32 at hydrotreating conditions. In an exemplary embodiment, hydrotreating conditions in the hydrotreater 30 are a temperature of from about 290° C. to about 400° C., and a pressure of from about 20 to about 140 atmospheres. The reaction conditions are generally more severe as the hydrotreating catalyst 32 ages and becomes less active, and for hydrocarbon streams with higher boiling points, where more severe reaction conditions include higher temperatures and/or pressures. The oxygen in the lignin 16 reacts with the hydrogen gas to produce water, and sulfur compounds or nitrogen compounds that may be present in the lignin aromatics stream 26 may also be reacted to form hydrogen sulfide and ammonia, respectively. The gases (including water, hydrogen sulfide, and ammonia, if present) are then separated from the propylbenzene containing feed 40, and excess hydrogen gas may be recovered and re-used in the hydrotreater 30.

In an exemplary embodiment, the hydrotreating catalyst 32 includes an IUPAC Group VI and/or IUPAC Group VIII active metal component on a support, where the support may be a porous refractory oxide including, but not limited to, alumina, alumina-silica, silica, zeolites, titania, zirconia, boria, magnesia, and their combinations. Supports other than refractory oxides are also possible in various embodiments. In some embodiments, other metals are included in the hydrotreating catalyst 32 in addition to or in place of the IUPAC Group VI and/or IUPAC Group VIII metals, such as cobalt, nickel, or other metals. For example, metals that may be used in the hydrotreating catalyst 32 include molybdenum, ruthenium, cobalt, nickel, tungsten, and combinations thereof. Hydrotreating catalysts 32 can be prepared by combining the active metals with the support. The supports, which may contain metal components, are typically dried and calcined at temperatures ranging from about 370° C. to about to 600° C. to eliminate any solvent and to convert metals to the oxide form, but other catalyst preparation processes are also possible. The calcined metal oxide catalysts may be reacted with sulfur to produce a metal sulfide, such as by contact with a sulfur containing compound including but not limited to hydrogen sulfide, organo sulfur compounds or elemental sulfur.

The lignin aromatics stream 26 may be the propylbenzene containing feed 40 in embodiments where no hydrotreater 30 is used. As such, the depolymerization conditions in the lignin reactor 22 may be relatively severe to produce the lignin aromatics stream 26 where oxygen is present at about 10 parts per million by weight or less. In an exemplary embodiment, the propylbenzene containing feed 40 is about 50 mass percent or more depolymerized lignin. In yet other embodiments, the propylbenzene containing feed 40 may be produced by reforming hydrotreated naphtha and/or pyrolysis gasoline feedstocks (not illustrated). The propylbenzene containing feed 40 may be produced by high-severity reforming that typically produces relatively high levels of higher alkyl substituted aromatic compounds, such as ethyl benzene, propyl benzene, and butyl benzene. Propylbenzene containing feeds 40 from a petroleum source may be hydrotreated in some embodiments, as described for the lignin aromatics stream 26 above. In yet other embodiments, the propylbenzene containing feed 40 is a combined stream with depolymerized lignin, reformed naphtha, reformed pyrolysis gasoline feedstocks, or other streams that include relatively high concentrations of higher alkyl substituted aromatic compounds. “Relatively high concentrations of higher alkyl substituted aromatic compounds,” as used herein, means streams where greater than about 25 mole percent of the A8 compounds are ethylbenzene, greater than about 10 mole percent of the A9 compound are propylbenzene, or greater than about 10 mole percent of the A10 compounds are butylbenzene. In some embodiments, the propylbenzene containing feed 40 includes about 40 mole percent or greater ethylbenzene, propylbenzne, and butylbenzene in the respective carbon number. The propylbenzene containing feed 40 includes other compounds in various embodiments, such as aromatic compounds with more than 10 carbon atoms.

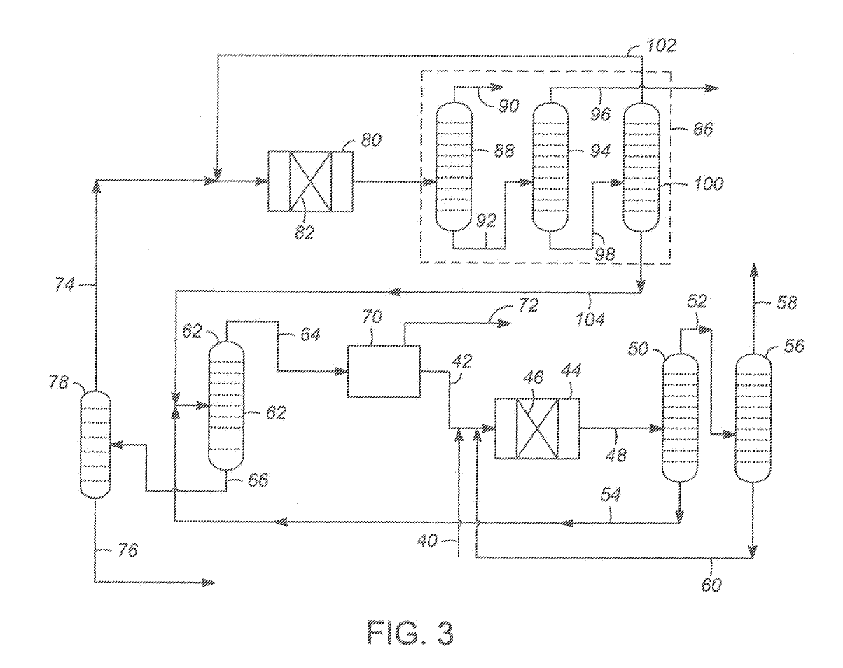

Reference is now made to the exemplary embodiment illustrated in FIG. 3. The propylbenzene containing feed 40 is combined with a xylene raffinate stream 42 and introduced to an isomerization unit 44 to produce a xylene isomerization effluent stream 48. The propylbenzene containing feed 40 and the xylene raffinate stream 42 may be combined before entering the isomerization unit 44 in some embodiments, but the propylbenzene containing feed 40 and the xylene raffinate stream 42 may also be combined after entering the isomerization unit 44 in other embodiments. The term “isomerization” is used herein to describe the conversion of an aromatic hydrocarbon into at least one different aromatic hydrocarbon having the same number of carbon atoms. Isomerization desirably converts those aromatics having at least one ethyl, propyl, or butyl substitute, such as methyl-ethyl benzene and propyl benzene into an aromatic compound with more methyl substitutes than before isomerization. The availability of additional methyl groups increases the xylene yield from the propylbenzene containing feed 40. In addition, the methyl groups on A9 compounds are highly stable at transalkylation reaction conditions, described below, and are essentially conserved in the transalkylation reaction. The higher alkyl benzene compounds are isomerized to form methylated aromatic compounds at equilibrium concentrations, or at closer to equilibrium concentrations than in the propylbenzene containing feed 40. In this description, “methylated aromatic compounds” means compounds that include a benzene ring with one or more attached methyl groups. The xylene raffinate stream 42 includes xylene isomers (A8 compounds), as discussed below, and the propylbenzene containing feed 40 includes A9-10 compounds. Therefore, the xylene isomerization effluent stream 48 also includes A8 and A9-10 compounds.

Different types of isomerization units exist, where some types of isomerization units generally cleave higher alkyl groups from benzene rings, and where other types of isomerization units (ethylbenzene isomerization units) generally form a more equilibrium mixture of aromatic compounds that include methylated aromatics. Therefore, if a feed stream includes a high concentration of propylbenzene, such as the propylbenzene containing feed 40, the ethylbenzene isomerization units tend to isomerize the propylbenzene and form methyl ethyl benzene and trimethyl benzene. The ethylbenzene isomerization units also tend to form methylated aromatic compounds with other higher alkyl aromatics when the higher alkyl aromatics enter the ethylbenzene isomerization unit at greater than the equilibrium concentration. In some embodiments, the isomerization unit 44 is an ethylbenzene isomerization unit, includes an isomerization catalyst 46, and is operated at isomerization conditions. The isomerization catalyst 46 can be in a fixed-bed system, a moving-bed system, a fluidized-bed system, or in a batch-type operation in the isomerization unit 44. The isomerization unit 44 may be one or more separate reactors configured to ensure that the desired isomerization temperature is maintained at the entrance to each reactor. The hydrocarbons from the propylbenzene containing feed 40 may contact the isomerization catalyst 46 in a liquid phase, a mixed liquid vapor phase, or in a vapor phase in various embodiments.

The hydrocarbons from the propylbenzene containing feed 40 and the xylene raffinate stream 42 contact the isomerization catalyst 46 at isomerization conditions, such as a temperature of from about 100° C. to about 500° C. and a pressure of from about 1 atmosphere to about 100 atmospheres. Sufficient isomerization catalyst 46 is contained in the isomerization unit 44 to provide a liquid hourly space velocity with respect to the hydrocarbon feed mixture of from about 0.1 to about 30 hr⁻¹ in some embodiments, and about 0.5 to about 10 h⁻¹ in other embodiments. Hydrocarbons from the propylbenzene containing feed 40 and the xylene raffinate stream 42 are reacted in admixture with hydrogen at a hydrogen/hydrocarbon mole ratio of about 0.5:1 to about 25:1 or more. ETHYLBENZENE type isomerization catalysts 46 are well known in the art, and many different types of ETHYLBENZENE isomerization catalysts 46 can be used. In one embodiment, the isomerization catalyst 46 includes an acid function to promote naphthenic ring transformation from cyclohexanes to cyclopentanes and back again; ethyl, propyl, and butyl group isomerization to substituted methyl groups; isomerization to a near-equilibrium distribution of aromatic isomers with the same carbon number (ortho-, meta-, and para-xylene); and a metal function to promote hydrogenation of aromatics and dehydrogenation of naphthenes. As such, the isomerization catalyst 46 may include a low Si/Al₂ MTW type zeolite, also characterized as “low silica ZSM-12,” with a silica to alumina ratio of about 60 or less to 1. The MTW-type zeolite may be composited with a binder for convenient formation of catalyst particles, where the binder may be a refractory inorganic oxide such as alumina. The isomerization catalyst 46 also includes a hydrogenation catalyst component, such as one or more of platinum, palladium, rhodium, ruthenium, osmium, and iridium (the platinum group metals), which may be present at about 0.01 to about 2 weight percent of the total isomerization catalyst 46. Other metals may also be present in various embodiments, including but not limited to tin, germanium, or lead. The metal component(s) may be in a variety of chemical forms, such as an oxide, sulfide, halide, oxysulfide, as an elemental metal, or in combination with one or more other ingredients of the catalyst composite.

Hydrocarbon compounds are partially saturated into naphthenic compounds in the isomerization unit 44, and carbon atoms on the higher alkyl groups move more freely in naphthenic compounds. The propylbenzene containing feed 40 has a high concentration of higher alkyl benzene compounds, as mentioned above, so the higher alkyl benzene compounds are present at more than the equilibrium concentration. As such, the xylene isomerization effluent stream 48 has a higher concentration of methylated aromatic compounds and a lower concentration of higher alkyl benzene compounds than the propylbenzene containing feed 40. The isomerization unit 44 may produce an equilibrium amount of naphthenes with the aromatic compounds, where the naphthenes are about 2 to about 20 mass percent of the xylene isomerization effluent stream 48, or about 4 to about 10 mass percent in other embodiments. The isomerization unit may produce the equilibrium amount of naphthenes regardless of the concentration of naphthenes in the feed, so if the xylene isomerization effluent stream 48 has an essentially constant concentration of naphthenes even if the propylbenzene containing feed 40 and xylene raffinate stream 42 have a higher or a lower concentration of naphthenes.

The A8 and A9-10 compounds in the xylene isomerization effluent stream 48 are separated, such as by fractionation. In an exemplary embodiment, a plurality of fractionators are used, but a single fractionator could be used in an alternate embodiment, or other combinations of fractionators could be used. The xylene isomerization effluent stream 48 may be introduced into a deheptanizer 50, and split into a deheptanizer light ends stream 52 and a deheptanizer heavy ends stream 54. The deheptanizer light ends stream 52 and deheptanizer heavy ends stream 54 are separated based on the relative boiling points of the components of the xylene isomerization effluent stream 48, and the deheptanizer light ends stream 52 may include non-aromatic compounds with 6 carbons and lighter compounds, where “lighter” refers to compounds with a boiling point less than a reference compound (non-aromatic compounds with 7 carbons in this example). As such the deheptanizer heavy ends stream 54 may include compounds heavier than non-aromatic compounds with 6 carbons, where “heavier” refers to compounds with a boiling point more than the reference compound (non-aromatic compounds with 7 carbons in this example). In this example, a heavy desorbent may be used to isolate a desired xylene isomer, as described below, and the dehexanizer 56 described below may not be used or included.

In an alternate embodiment where a light desorbent is used in the xylene recovery unit 70, the deheptanizer light ends stream 52 may include toluene, non-aromatic compounds with 8 carbons, and lighter compounds. The deheptanizer light ends stream 52 is then fractionated in a dehexanizer 56 to produce a dehexanizer light ends stream 58 and a dehexanizer heavy ends stream 60. The dehexanizer light ends stream 58 may include non-aromatic compounds with 6 carbons and lighter compounds, and the dehexanizer heavy ends stream 60 may include toluene and non-aromatic compounds with 7 or 8 carbons. The dehexanizer heavy ends stream 60 may be combined with the propylbenzene containing feed 40 and the xylene raffinate stream 42 and recycled to the isomerization unit 44. The dehexanizer 56 allows for toluene to be recycled through the isomerization unit 44 without entering the xylene recovery unit 70 described below. Toluene may be the light desorbent used in the xylene recovery unit 70, so separating toluene from the xylene recovery unit 70 may be beneficial. The dehexanizer light ends stream 58 or the deheptanizer light ends stream 52 with non-aromatic compounds having 6 carbons or less is discharged, and may be used or further refined into paraffinic solvents, blended into gasoline, used as a feedstock for an ethylene plant, or otherwise used in a variety of manners.

The deheptanizer heavy ends stream 54, which includes A8 compounds and A9-10 compounds, is transferred to a xylene column 62 where it is separated into a first xylene column stream 64 and second xylene column stream 66. The first xylene column stream 64 includes xylenes, or A8 compounds, and the first xylene column stream 64 is introduced into the xylene recovery unit 70. The xylene recovery unit 70 separates a xylene isomer stream 72 from the xylene raffinate stream 42, as described below, where the xylene isomer stream 72 includes a desired xylene isomer. Several techniques can be used for the xylene recovery unit 70, including selective adsorption and desorption, simulated moving bed adsorption, crystallization, ionic liquid separation, selective membranes, Etc. In an exemplary embodiment using selective adsorption and desorption using a simulated moving bed, the xylene isomer stream 72 includes about 99% or greater para-xylene, along with the desorbent that can be separated from the para-xylene by distillation. The xylene raffinate stream 42 is almost entirely depleted of para-xylene, or the desired xylene isomer in embodiments where an isomer other than para-xylene is desired. The desorbent is recovered from the xylene isomer stream 72 and the xylene raffinate stream 42 by distillation (not illustrated), as understood by those skilled in the art. The desorbent may be heavier or lighter than the xylenes in various embodiments. In an exemplary embodiment, the xylene recovery unit 70 includes one or more adsorbent chambers with a selective adsorbent that preferentially adsorbs the desired xylene isomer over the other xylene isomers. In an exemplary embodiment, the selective adsorbent can be crystalline alumino-silicate, such as type X or type Y crystalline aluminosilicate zeolites. The selective adsorbent contains exchangeable cationic sites with one or more metal cations, where the metal cations can be one or more of lithium, potassium, beryllium, magnesium, calcium, strontium, barium, nickel, copper, silver, manganese, and cadmium. Adsorption conditions vary, but the temperature typically ranges from about 35° C. to about 200° C. and the pressure may be from about 1 atmosphere to about 35 atmospheres. The xylene raffinate stream 42 is transferred to the isomerization unit 44, as described above, to create a loop for the xylene isomers other than the desired xylene isomer.

The second xylene column stream 66 is optionally separated into an A9-10 stream 74 and an A11+ stream 76 in a heavy aromatics column 78. The A11+ stream 76 includes aromatic compounds with 11 or more carbons (A11+ compounds), and the A9-10 stream 74 includes A9-10 compounds. The A9-10 stream 74 may also include some naphthenes that are heavier than xylene. The A9-10 stream 74 is transferred to a transalkylation unit 80, where it is contacted with a transalkylation catalyst 82 in the presence of toluene at transalkylation conditions.

The process continues by transalkylating the A9-10 stream 74, where the A9-10 stream 74 includes methylated A9-10 compounds. The A9-10 stream 74 and toluene are contacted with a transalkylation catalyst 82 in the transalkylation unit 80 at transalkylation conditions to produce a transalkylation effluent stream 84. The transalkylation unit 80 disproportionates toluene into benzene and mixed xylenes and transalkylates the methyl-enriched A9-10 compounds from the A9-10 stream 74 into xylenes and benzene, as known in the art. The transalkylation unit 80 produces mixes xylenes from the toluene and A9-10 compounds, and the mixed xylenes are further processed to produce a desired xylene isomer, as described above. The transalkylation unit tends to cleave higher alkyl groups from benzene, so any ethyl, propyl, or butyl benzene compounds that enter the transalkylation unit may be converted to benzene and the associated paraffin or naphthene. However, the propylbenzene containing feed 40 has been isomerized and methylated in the isomerization unit 44 prior to being transalkylated, and methylated A9 and A10 compounds may form xylene in the transalkylation unit 88.

The transalkylation effluent stream 84 passes to a fractionation zone 86 where the various components are separated. In an exemplary embodiment, the fractionation zone 86 includes a transalkylation stripper 88, and benzene column 94, and a toluene column 100, but other embodiments of the fractionation zone 86 are also possible. The transalkylation stripper 88 produces a transalkylation stripper vapor stream 90 and a transalkylation stripper liquid stream 92, where the transalkylation stripper vapor stream 90 includes non-aromatic compounds with 5 carbons or less, and the transalkylation stripper liquid stream 92 includes compounds that are heavier than non-aromatic compounds with 5 carbons. The transalkylation stripper liquid stream 92 is transferred to the benzene column 94, where the benzene column 94 produces a benzene stream 96 including benzene, and an A7+ stream 98 including A7+ compounds. In an exemplary embodiment, the benzene stream 96 includes about 90 mass percent or more benzene. The A7+ stream 98 is provided to the toluene column 100, where it is separated into a toluene stream 102 including toluene, and an A8+ stream 104 including A8+ compounds. In an exemplary embodiment, the toluene stream 102 includes about 90 mass percent or more toluene. The toluene stream 102 may be combined with the A9-10 stream 74, either before or within the transalkylation unit 80, to provide the toluene for transalkylation with the A9-10 compounds in the A9-10 stream 74. A8 compounds are separated from toluene in the toluene column 100, and A8 compounds are separated from A9-10 stream 74 in the xylene column 62, so there may be very little (less than about 5 mass percent) xylene in the feed to the transalkylation unit 80. The A8+ stream 104 is provided to the xylene column 62, where the A8 compounds (xylene) are separated and included in the first xylene column stream 64.

The apparatus and method described above allows for the production of a desired xylene isomer from a propylbenzene containing feed 40, where the propylbenzene containing feed 40 may be produced from a renewable resource. The renewable resource, lignin, is also used to produce benzene and various other products, as described above. Other feedstocks may be co-processed with the propylbenzene containing feed 40 in some embodiments (not illustrated), such as an aromatic petroleum feedstock including a petroleum reformate, as understood by those skilled in the art. As such, the aromatic petroleum feedstock, or components thereof, is processed in the xylene recovery unit 70 to isolate the desired xylene isomer.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

What is claimed is:

1. A method of producing xylene comprising:
combining a propylbenzene containing feed with a xylene raffinate stream, wherein the xylene raffinate stream is provided from a xylene recovery unit;
isomerizing the xylene raffinate stream and the propylbenzene containing feed in an isomerization unit to produce an xylene isomerization effluent stream comprising aromatic compounds having 8 carbon atoms (A8 compounds) and aromatic compounds having 9 to 10 carbon atoms (A9-10 compounds);
separating the A8 compounds from the A9-10 compounds;
feeding the A8 compounds to the xylene recovery unit; and
transalkylating the A9-10 compounds with toluene to produce xylene.

2. The method of claim 1 further comprising:
depolymerizing a lignin supply to produce the propylbenzene containing feed.

3. The method of claim 2 wherein depolymerizing the lignin supply to produce the propylbenzene containing feed comprises depolymerizing the lignin supply to produce about 50 mass percent or more of the propylbenzene containing feed.

4. The method of claim 2 wherein depolymerizing the lignin supply comprises:
forming a lignin slurry from the lignin supply and a lignin solvent, wherein the lignin solvent comprises about 50 mass percent water or more; and
contacting the lignin slurry with a lignin catalyst at depolymerization conditions.

5. The method of claim 2 further comprising:
reducing an oxygen content of the propylbenzene containing feed to about 10 parts per million or less by weight prior to isomerizing the propylbenzene containing feed.

6. The method of claim 1 further comprising:
hydrotreating the propylbenzene containing feed prior to isomerizing the propylbenzene containing feed.

7. The method of claim 1 wherein separating the A8 compounds from the A9-10 compounds comprises fractionating the xylene isomerization effluent stream to produce a first xylene column stream comprising the A8 compounds and a second xylene column stream comprising the A9-10 compounds.

8. The method of claim 7 further comprising:
separating the second xylene column stream into an A9-10 stream and an A11+ stream, wherein the A9-10 stream comprises the A9-10 compounds and the A11+ stream comprises aromatic compounds having 11 carbon atoms or more; and
wherein transalkylating the A9-10 compounds comprises feeding the A9-10 stream to a transalkylation unit.

9. The method of claim 7 further comprising:
separating a desired xylene isomer from the first xylene column stream in the xylene recovery unit.

10. The method of claim 9 wherein separating the desired xylene isomer comprises separating para-xylene.

11. The method of claim 1 wherein transalkylating the A9-10 compounds with toluene comprises producing a transalkylation effluent stream, the method further comprising:
fractionating the transalkylation effluent stream in a fractionation zone to produce a benzene stream and a toluene stream, wherein the benzene stream comprises about 90 mass percent or more benzene and the toluene stream comprises about 90 mass percent or more toluene.

12. The method of claim 11 further comprising:
combining the toluene stream with the A9-10 compounds in a transalkylation unit.

13. The method of claim 1 further comprising:
introducing an aromatic petroleum feedstock into the xylene recovery unit.

14. The method of claim 1 wherein isomerizing the xylene raffinate stream and the propylbenzene containing feed comprises contacting the xylene raffinate stream and the propylbenzene containing feed with an isomerization catalyst, wherein the isomerization catalyst comprises a zeolite with a silica to alumina ratio of about 60 or less to 1, and wherein the isomerization catalyst comprises a platinum group metal in an amount of from about 0.01 to about 2 weight percent of the isomerization catalyst.

15. The method of claim 1 further comprising:
stripping nonaromatic hydrocarbons having 5 carbon atoms or less from a transalkylation effluent stream to produce a transalkylation stripper liquid stream, wherein the transalkylation effluent stream is produced from transalkylating the A9-10 compounds with toluene; and
fractionating the transalkylation stripper liquid stream to produce a benzene stream, wherein the benzene stream comprises about 90 mass percent or more benzene.

16. The method of claim 1 further comprising:
separating a deheptanizer light ends stream from the xylene isomerization effluent stream to produce a deheptanizer heavy ends stream, wherein the deheptanizer light ends stream comprises non-aromatic compounds having 6 carbon atoms or less, and the deheptanizer heavy ends stream comprises the A8 compounds and the A9-10 compounds.

17. A method of producing xylene comprising:
isolating a desired xylene isomer from a first xylene column stream in a xylene recovery unit, and producing a xylene raffinate stream;
contacting the xylene raffinate stream and a propylbenzene containing feed with an isomerization catalyst at isomerization conditions to produce a xylene isomerization effluent stream comprising aromatic compounds having 8 carbon atoms (A8 compounds) and aromatic compounds having 9 to 10 carbon atoms (A9-10 compounds);
separating the A8 compounds from the A9-10 compounds; and
contacting the A9-10 compounds with a transalkylation catalyst at transalkylation conditions, and in the presence of toluene, to produce xylene.

18. The method of claim 17 wherein contacting the xylene raffinate stream and the propylbenzene containing feed with the isomerization catalyst comprises contacting the propylbenzene containing feed with the isomerization catalyst wherein the propylbenzene containing feed comprises depolymerized lignin.

19. The method of claim 17 wherein separating the A8 compounds from the A9-10 compounds comprises:
fractionating the xylene isomerization effluent stream to produce the first xylene column stream comprising the A8 compounds, an A9-10 stream comprising the A9-10 compounds, and an A11+ stream comprising aromatic compounds having 11 or more carbon atoms (A11+ compounds).

20. An apparatus for producing xylene comprising:
a xylene recovery unit configured to isolate a desired xylene isomer from a first xylene column stream;
an isomerization unit coupled to the xylene recovery unit;
a propylbenzene containing feed coupled to the isomerization unit;
a transalkylation unit configured to transalkylate aromatic compounds having 9 or more carbon atoms with toluene to produce xylene;
a xylene column coupled to the xylene recovery unit, wherein the xylene column is configured to transfer the first xylene column stream to the xylene recovery unit and to transfer a second xylene column stream to the transalkylation unit; and
a fractionation zone coupled to the transalkylation unit and to the xylene column, wherein the fractionation zone is configured to transfer aromatic compounds having 8 or more carbons to the xylene column.
